(19) United States
(12) Patent Application Publication
(10) Pub. No.: US 2006/0035369 A1
(43) Pub. Date: Feb. 16, 2006

(54) Sample support washing container, sample support washing station, system for washing sample slides and method for washing sample slides

(76) Inventors: Christoph Gauer, Munchen (DE); Jurgen Scriba, Munchen (DE); Joachim Arzt, Neubiberg (DE); Roland Kirchner, Edling (DE); Andreas Togl, Haar (DE)

Correspondence Address: DILWORTH & BARRESE, LLP, 333 EARLE OVINGTON BLVD., UNIONDALE, NY, 11553, US

(21) Appl. No.: 11/197,142

(22) Filed: Aug. 4, 2005

(30) Foreign Application Priority Data
Aug. 4, 2004 (DE) 102004037848.7

(51) Int. Cl. C12M 1/34

(52) U.S. Cl. 435/287.1

(57) ABSTRACT

The invention relates to a sample support washing container for simultaneous washing of at least two sample slides, with a filling opening, through which the sample slides can be introduced, a liquid supply opening or respectively discharge opening substantially opposite the filling opening and sample slide holding devices, arranged in the washing container such that they can keep the introduced sample slides apart, and a liquid discharge space remains between the introduced sample slides and the liquid supply opening or respectively discharge opening. The invention furthermore relates to a sample support washing station for washing sample slides, with at least one take-up for receiving an inventive sample support washing container, whereby the take-up has a station supply/discharge, which corresponds to its liquid supply opening or respectively discharge opening when the sample support washing container is removed, an overflow vessel arranged around the take-up, a pump connected to the station supply/discharge and at least one liquid supply valve connected to the pump. Finally, the invention relates to a system for washing sample slides with an inventive sample support washing station and at least an inventive sample support washing container and a method for washing and drying sample slides using an inventive sample support washing station and at least an inventive sample support washing container.

The invention relates to a sample support washing container for washing sample slides with microarrays or tissue slices, in particular microscope slides, whereby the sample support washing container has a filling opening, through which sample slides can be introduced. The invention furthermore relates to a sample support washing station for washing sample slides with microarrays or tissue slices, in particular microscope slides, a system for washing sample slides using such sample support washing containers and such a sample support washing station and a method for washing and drying sample slides using such sample support washing containers and such a sample support washing station.

So-called DNA microarrays are frequently employed these days to examine macromolecules. At the same time different types of macromolecules are arranged in spots at different places e.g. on a microscope slide in a uniform arrangement. Such microarrays are frequently applied to microscope slides, which have approximately the format 75 mm×25 mm. A liquid with another type of macromolecules is put in contact with the microarray. The macromolecules of the liquid do not make specific bonds with all macromolecules of the matrix spots, so that from those places where a specific binding or respectively hybridising has taken place, information can be obtained about the type of macromolecules in the liquid (DNA Microarrays, A Practical Approach, edited by M. Schena, Oxford University Press, 1999, Chapter 1.3, pages 6 to 12).

Following hybridising of DNA microarrays or also following incubation of protein microarrays these must be washed thoroughly, so that unspecific bonds are removed and the signal-to-noise ratio is accordingly improved. Typical washing protocols comprise several steps, as described here e.g. for DNA microarrays (DNA Microarrays, M. Schena, page 11):
1. wash the array in 1×SSC and 0.1% SDS at room temperature for 5 minutes (400 ml volume)
2. wash the array 0.1×SSC and 0.1% SDS at room temperature for 5 minutes (400 ml volume)
3. immerse the array briefly in 0.1×SSC at room temperature to wash off SDS (400 ml volume).

As specified by way of example in this protocol, washing is frequently carried out in solutions with increasing salt concentration. The arrays are then dried to prevent the development of spurious salt crusts and water streaking. The microarrays are generally centrifuged or also discharged at ca. 600×g (600×gravitational acceleration) for drying.

There are also similar washing steps such as described hereinabove in the area of FISH (fluorescence in situ hybridisation) and IHC (immunohistochaemia). Here, too, microscope slides, to which tissue slices or respectively cell suspensions are applied, in general serve as substrate.

The microscope slides are as a rule washed by hand these days. For this the arrays are set in so-called trays after incubation. Next the tray is dipped by hand in containers with different washing solutions. Several arrays can be washed in parallel with the tray. Errors, which impair the reproducibility of the results, can result from the manual working of the protocol. Small irregularities in handling can also lead to the fact that streaking or impurities remain on the sample, which then make evaluation of the signals difficult, or falsify the outcome.

During washing the tray is shifted from side to side to remove the unspecifically bound molecules from the substrate surface. The resulting mixture can be reproduced only poorly, with the consequence that the biological results likewise reproduce poorly.

U.S. Pat. No. 6,238,910 B1 describes an automatic hybridising station, in which incubation and subsequent washing are carried out automatically. The sample to be examined is hybridised in a capillary slot on an array, which is applied to a substrate in the format of a microscope slide. Washing solutions are pumped through the capillary slot, whereby the flow rate can be adjusted. Incubation or respectively hybridising should occur in a very small volume, according to a high concentration at a small quantity of starting material, to achieve the best possible signal-to-noise ratio. By comparison, the washing should happen in a large volume, since here a high degree of dilution is advantageous. IN the method described in U.S. Pat. No. 6,238,910 B1 both these steps take place in the same chamber, so that a compromise between these requirements must be found. In appliances, currently on the market, the required sample volume is as a rule between 100 μl and 200 μl and is thus approximately twice as high as with manual hybridising. The washing volume with manual hybridising on the other hand is above 100 ml and is thus higher by orders than in these appliances. In addition, with these appliances there is the risk that the fluidic elements of the appliances, which as a rule have a diameter of 1 mm or less, easily become encrusted or clog up, because solutions containing salt are often used.

U.S. Pat. No. 5,595,707 describes an instrument, by means of which arrays or tissue slices are incubated and then washed. The microscope slides are arranged on a carousel. The sample or respectively probe fluid is present as an open drop, which can be covered by oil when required to prevent evaporation during incubation. The washing step is performed by means of a nozzle which sprays off the microscope slide. As a rule the oil cannot be fully eliminated through the nozzle. A manual washing step for complete removal of any oil residue is necessary.

The drawback to appliances currently available on the market is that incubation and washing are performed in the same chamber. The automation made available with fluidics is therefore utilised only very inefficiently. DNA microarrays are generally incubated overnight, while the washing lasts only a few minutes. Due to expensive fluidics however a majority of appliance costs (pumps, valves, hoses and couplings) is attributed to automating of the washing procedure, which as a rule is carried out only once per day for a few minutes.

The object of the present invention is to provide cost-effective devices and methods for washing sample slides for easy and reproducible washing.

This task is solved by a sample support washing container with the characteristics of Claim 1, a sample support washing station with the characteristics of Claim 10, a system for washing sample slides with the characteristics of Claim 21 and a method for washing and drying sample slides with the characteristics of Claim 22. Independent claims focus on advantageous embodiments.

In particular an inventive sample support washing container has a liquid supply opening or respectively discharge opening, which is arranged substantially opposite the filling opening, through which the sample slides are introduced. Inside the sample support washing container sample slide holding devices are provided, which are arranged such that they hold the introduced sample slides apart from one another, and a liquid discharge space remains between the introduced sample slides and the liquid supply opening or respectively discharge opening. For example, microarrays, microscope slides for IHC and FISH or proprietary array formats can be washed with such sample support washing containers. The washing volume is adequate to ensure strong dilution. One or more washing/rinsing solutions can be rinsed by the opposite filling opening and liquid supply opening or respectively discharge opening through the sample support washing container. The sample slide holding devices enables several sample slides to be held in a defined position, in the sample support washing container, so that a side of the microscope slide is free. It is therefore possible, using the inventive sample support washing container, to treat a number of sample slides at the same time.

In the present text the terms “washing solution” and “rinsing solution” are used synonymously. Also the terms “washing” and “rinsing” are used synonymously.

It is particularly possible for the sample slides to be rinsed by the two opposite openings of the sample support washing container. A static liquid level, as with cleaning devices of the prior art, can thus be avoided. Especially in liquids containing salt a static liquid level hides the risk of encrusting where the liquid level touches the edge of the sample support washing container. If the inventive sample support washing container is used e.g. in a centrifuge to remove residual fluid residue, this liquid gathers in the sample slides discharge space or flows out of there into a separate container or the centrifuge. The liquid discharge space thus effectively prevents centrifuged fluid residue from spraying back onto the sample slides.

Sample support washing containers, which are designed cylindrical, e.g. with the external dimensions of a 50 ml centrifuge tube or respectively a Falcon tube, whereby the filling opening and the liquid supply opening or respectively discharge opening are on the opposite cylinder faces, are particularly easy to handle and flexible in use. The sample slide holding devices are appropriately arranged such that the sample slides are held substantially parallel to the cylindrical axis.

A particularly advantageous configuration of the inventive sample support washing container provides that its external dimensions correspond to the standard dimensions for 50 ml centrifuge tubes or respectively Falcon tubes to be used in conventional centrifuges. With such sample support washing containers it is easily possible to perform a drying step in a conventional centrifuge following the washing procedure. The sample support washing container with the sample slides in it can be placed directly in a centrifuge, without transfer of the sample slides being necessary, and are dried there by centrifuging. The liquid flowing out during centrifuging gathers in the liquid discharge space, which remains between the sample slides and the liquid supply opening or respectively discharge opening, without further touching the sample slides, or respectively the liquid flows into the centrifuge or a separate collecting container.

The liquid discharge space situated between liquid supply opening or respectively discharge opening and sample slides can have various forms. A conical configuration is particularly beneficial, in the tip whereof the liquid supply opening or respectively discharge opening is located. Such a configuration allows a liquid supply or respectively liquid discharge to take place securely and in a defined manner. Also, a conical configuration effectively prevents liquid residue centrifuged from the sample slides in the centrifuge from being able to spray back onto the sample slide surface.

A simple configuration of the sample support washing container has ribs pointing radially inwards as sample slide holders. Corresponding sample slides can easily be slipped in between such ribs through the filling opening.

The sample support washing container can comprise e.g. glass, is A sample support washing container made of plastic is easy to produce and robust.

An inventive sample support washing station for washing sample slides has at least one take-up, suitable for taking up at least one inventive sample support washing container. The at least one take-up of the sample support washing station has a station supply/discharge, which corresponds to its liquid supply opening or respectively discharge opening when the sample support washing container is taken up. An overflow vessel is arranged around the at least one take-up. Finally, the inventive sample support washing station has a pump connected to the station supply/discharge and at least one liquid supply valve attached to the pump.

Inventive sample slides are easily used with such a sample support washing station. Washing solution can be sluiced through the sample support washing container by the station supply/discharge, by means of the pump. The liquid rises out of the sample support washing container and trickles into the inventive overflow. In this way contamination from one washing solution to the other is effectively prevented.

The inventive overflow vessel can be sufficiently big to be able to take up typically occurring quantities of liquid. However, there is an embodiment which is particularly advantageous, in which a discharge from the overflow vessel is provided, which can be utilised to empty the overflow vessel.

An individual valve can be provided for the liquid supply. Particularly versatile however is a configuration, in which a valve block with several supply valves through which different washing liquids can be introduced to the system, is provided for liquid supply upstream of the pump. A liquid discharge pipe is advantageously provided between the at least one supply valve and the pump. When the pumping direction is reversed liquid can be drawn off through this discharge pipe.

The sample support washing station can be designed to use an individual inventive sample support washing container. It is particularly advantageous however if the sample support washing station can take up several sample support washing containers, which can be filled or respectively emptied. With an appropriate configuration these take-ups are grouped together such that they are positioned inside a common overflow vessel.

To enable favourable take-up of an inventive sample support washing container with a conical liquid supply opening or respectively discharge opening, the station supply/discharge is likewise designed conical in an advantageous further development of the inventive sample support washing station.

The take-up of the inventive sample support washing station is designed advantageously cylindrical to enable secure holding of cylindrical sample support washing containers. The take-ups should beneficially have inner measurements, which substantially have the external dimensions of centrifuges used in conventional standard vessels, in particular 50 ml centrifuge tubes, to ensure optimal compatibility. With such a configuration inventive sample support washing containers can are placed directly by the sample support washing station into conventional centrifuges for drying, without the sample slides having to be touched directly.

In an inventive method for washing and drying sample slides the sample slides are set in an inventive sample support washing container with microarrays or tissue slices, in particular microscope slides. The sample support washing container is placed in the take-up of an inventive sample support washing station. At least one washing liquid is rinsed by the sample support washing container through the station supply/discharge of the take-up of the sample support washing station and through the liquid supply opening or respectively discharge opening of the sample support washing container. After the washing/rinsing with at least one washing liquid the sample support washing container is placed in a centrifuge, without the sample slides being taken out of the sample support washing container. The sample slides are dried by centrifuging the sample slides in the centrifuge. During centrifuging excess liquid, remaining on the sample slides, flows in the liquid discharge space or via the liquid supply opening or respectively discharge opening in the centrifuge. Alternatively, a separate collecting container can e.g. be provided, which is set from below onto the sample support washing container e.g. for centrifuging. Through using the inventive sample support washing container and the inventive sample support washing station secure rinsing of the sample slides with washing liquid is ensured. The sample support washing containers can be transferred directly from the sample support washing station to a centrifuge, without the sample slides having to be removed from the sample support washing container.

At the same time the individual steps can be carried out in a different sequence, depending on requirements and liquids used. In order to securely prevent the hybridising solution from drying on the sample slides, before the washing process is finished, first the sample support washing container can be placed in the sample support washing station and a first washing/rinsing solution can be introduced to the sample support washing container via the station supply/discharge of the take-up of the sample support washing station and the liquid supply opening or respectively discharge opening of the sample support washing container. In the sample support washing container already filled with liquid The sample slides are placed in directly from the hybridising solution.

In another method the sample slides are first placed in the sample support washing container and the sample support washing container is set in the take-up of the sample support washing station either before or after the sample slides are adjusted. Only then is washing or respectively rinsing liquid rinsed through the station supply/discharge of the take-up of the sample support washing station and the liquid supply opening or respectively discharge opening of the sample support washing container. In the case of this method it is ensured that all sample slides come in contact with the washing or respectively rinsing solution at exactly the same time.

By using a correspondingly arranged sample support washing station it is possible with the inventive method to use different washing liquids with different wash cycles or temperatures.

It is particularly advantageous with the inventive method if a “mixing procedure” is undertaken through brief operation of the pump. At the same time, through brief switching on of the pump e.g. after complete exchange of the washing solution either air or a further washing solution can be introduced to cause thorough mixing.

Reproducible washing results can be achieved with the inventive sample support washing containers, the inventive sample support washing station, the inventive system or respectively with the inventive method. Hybridising or respectively incubation and washing happen in separate appliances, enabling better machine usage than in known fully automatic machines. The large washing volume leads to stronger and faster dilution, whereby the washing process is influenced favourably. Bubble-free/foam-free washing is also possible using solutions containing detergents and the overflow vessel.

The invention will now be explained in greater detail by means of the attached figures, which illustrate an embodiment of an inventive sample support washing container and an inventive sample support washing station, in particular in which:

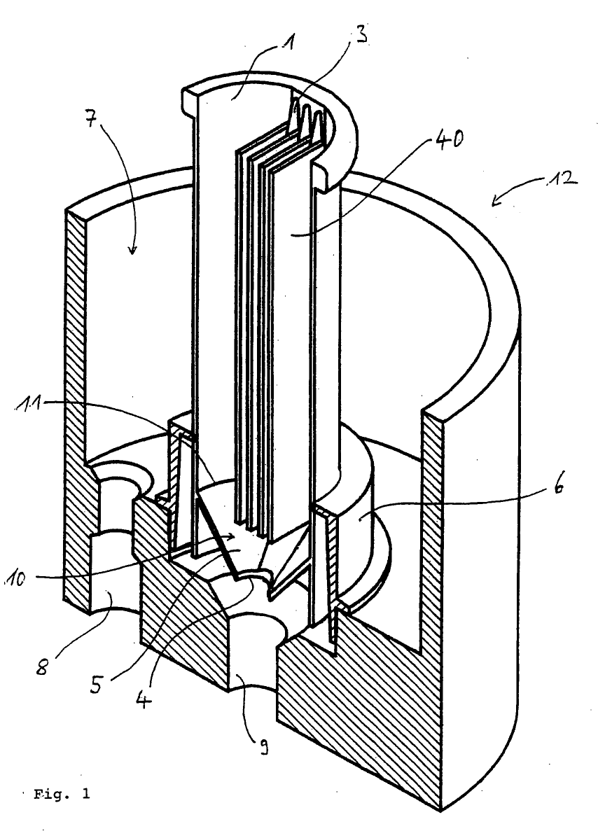

FIG. 1 shows the cross-section through the washing chamber of an inventive sample support washing station with an inventive sample support washing container,

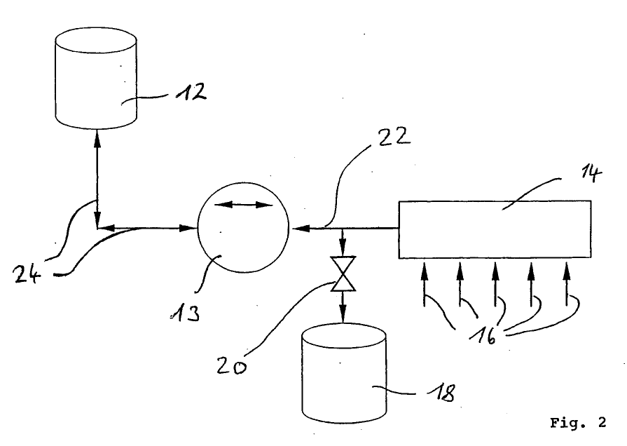

FIG. 2 shows a block diagram of an inventive sample support washing station,

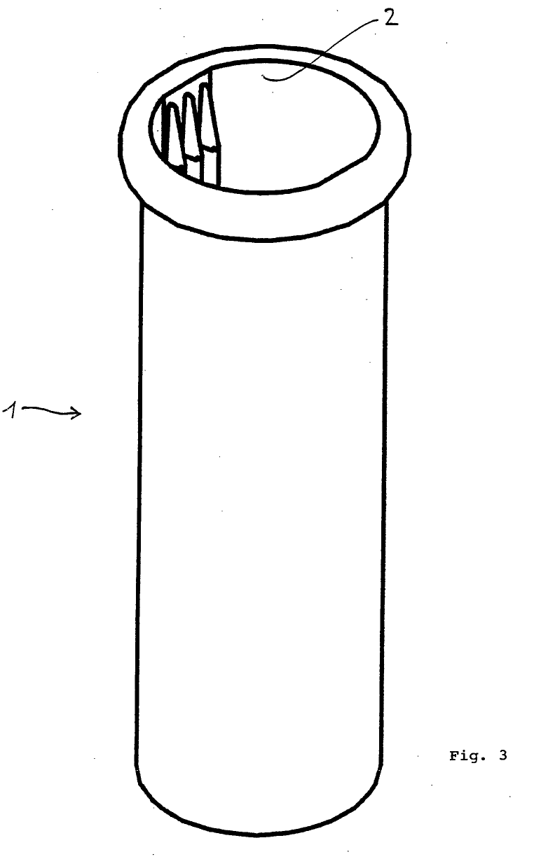

FIG. 3 shows the outer view of an inventive sample support washing container, and

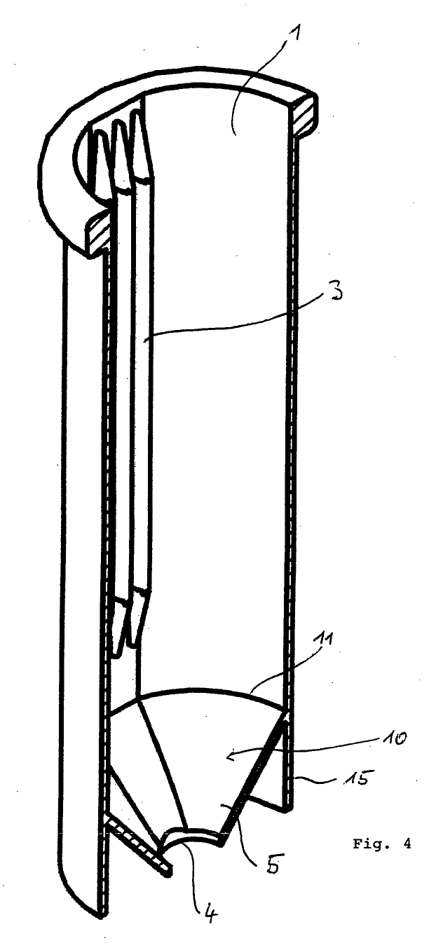

FIG. 4 shows the cross-section through an inventive sample support washing container.

The sample support washing container 1 shown in FIG. 3 has an interior 2, which has e.g. 50 ml volume, if it is suited for washing four standard slide carriers. In this embodiment the inner diameter of the sample support washing container 1 is approximately as large as the short side of a standard slide carrier. In the embodiment illustrated the measurements of the sample support washing container allow the placing of standard slide carriers having a width of 24.3 mm to 26 mm and a length of 74 mm to 75 mm. The illustrated sample support washing container is an injection-moulded item made of polypropylene.

FIG. 4 shows a cross-section. Evident here are the sample slide ribs 3, which are arranged opposite in the sample slide 1. These are inwards pointing ribs, between which microscope slides can be pushed. Located in the lower region of the sample support washing container is a conical tapering 5, terminating in the liquid supply opening or respectively discharge opening 4. Reference numeral 10 designates a liquid discharge space, which corresponds to the inner volume of the cone 5. On the line 11 the cone intersects the mantle surface of the sample support washing container 1. Arranged around the conical lower termination is a collar 15, which guarantees secure standing of the sample support washing container 1 both in an inventive sample support washing station and also at another site.

Microscope slides slid in between the ribs 3 are supported on the line 11 against the cone 5 and cannot slip further into the sample support washing container, and the liquid discharge space 10 thus remains free.

FIG. 1 shows an inventive sample support washing container 1 as an inventive sample support washing station is being placed in the washing chamber 12. The washing chamber 12 has a take-up 6, whereof the inner measurements correspond to the external dimensions of the sample support washing container 1. Both these measurements advantageously correspond approximately to the external dimensions of a 50 ml centrifuge tube, which can also be placed in a conventional centrifuge.

Here the take-up 6 is designed as a separate part, which is placed in the washing chamber 12. It can comprise e.g. plastic. In other embodiments the washing chamber 12 and the take-up 6 are made of one piece. Depending on requirement additional sealing elements can be provided between the sample support washing container 1 and the take-up 6.

The liquid supply opening or respectively discharge opening 4 of the cone 5 of the sample support washing container 1 is located opposite the station supply/discharge 9, which is likewise conical configured in the illustrated embodiment to ensure secure liquid discharge. Evident in the sectional view of FIG. 1 furthermore are four microscope slides 40 set into the sample support washing container 1, on which a microarray, not shown here, is positioned.

Arranged around the take-up 6 is an overflow 7, which receives liquid flowing out through the upper filling opening of the sample support washing container. The overflow 7 has a discharge 8, which e.g. can correspondingly have a pipe for discharging excess liquid.

FIG. 2 shows the connection diagram of an inventive sample support washing station. The washing chamber 12 is connected via pipes 24 to a hose pump 13, which can supply in both directions. The hose pump 13 is connected to a valve block 14 via pipes 22, in which supply pipes 16 for different washing or respectively rinsing solutions terminate. The valves of the valve block can be used to make a selection from these supply pipes 16. Arranged between valve block 14 and hose pump 13 is a branching for liquid waste. This waste pipe terminates in a liquid waste reservoir 18 by way of a valve 20.

The inventive device as described can be used as follows. By way of example the washing and drying of microscope slides with a microarray set thereon for removing unspecifically bound macromolecules following a hybridising step is described.

At the beginning, according to a first method the microscope slides 40, after hybridising, which is not an object of the present text and as such is not described in greater detail, are slipped out of the hybridising station into a sample support washing container 1 through the upper filling opening between the inwards pointing ribs 3. In the illustrated example four microscope slides can be inserted. When the microscope slides are inserted in between the ribs 3 the microscope slides are supported on the edge 11 on the cone 5. With the microscope slides 40 it contains the sample support washing container 1 is set in the take-up 6 of a washing chamber 12 of an inventive sample support washing station.

According to an alternative method the sample support washing container 1 is first set in the take-up 6 of the sample support washing station. A first washing/rinsing solution is introduced to the interior of the sample support washing container via the station supply/discharge 9 and the liquid supply opening or respectively discharge opening 4. An optical liquid sensor, not illustrated here, can be provided e.g. in the vicinity of the station supply/discharge 9 to predetermine the amount of liquid to be introduced to the washing chamber 12. The liquid sensor detects the influx of the liquid. A defined volume is conveyed to the washing chamber 12 via the discretionary replenishing time of the hose pump 13. Only then is the sample slide 40 brought directly from the hybridising solution to the sample support washing container 1 now filled with liquid. With this method there is no danger that the hybridising solution has already dried onto one or more sample slides 1, before it comes into contact with the first rinsing solution.

Following this and after a desired washing or rinsing liquid is selected in both described cases in the valve block 14 the corresponding valve for a supply pipe 16 is opened. The liquid is forwarded to the washing chamber 12 by means of the pump 13. The liquid flows through the station supply/discharge 9 and the liquid supply opening or respectively discharge opening 4 into the interior of the sample support washing container 1. It flows round the arranged microscope slides 40 with the microarrays arranged thereon, flows upwards from the sample support washing container 1 and is collected in the overflow vessel 7 or immediately discarded. So as to ensure thorough washing of the microscope slides a maximum volume of 60 ml solution is to be supplied per washing procedure in the illustrated embodiment. Foam resulting from liquids containing detergent is likewise carried away via the overflow.

Approximately 5 ml washing solution is supplied during the washing procedure with the washing solution per discretionary time interval to simulate the manual mixing motion. A mixing procedure is simulated by the intermittently supplied washing solution.

After the washing procedure the remaining rinsing solution can be pumped by means of the pump 13 out of the washing chamber and fed to the liquid waste reservoir 18 on opening of the valve 20.

On completion of a washing procedure another liquid can be utilised through selecting another valve in the valve block 14.

The washing procedure can be carried out automatically e.g. by means of a microprocessor control for the pump and the valves.

After the washing or respectively rinsing steps have correspondingly been carried out the sample support washing container 1 is taken out of the washing chamber 12 with the microscope slides also contained therein and placed in a conventional centrifuge. Removing the microscope slides from the sample support washing container 1 is not necessary. In the centrifuge the liquid is centrifuged from the microscope slides 40 and flows through the liquid discharge space 10 and the liquid supply opening or respectively discharge opening 4 into the centrifuge.

In an embodiment or respectively method not shown here a separate collection container is set on the sample support washing container 1 from below, which can be e.g. cylindrical, for catching the liquid flowing out through the liquid discharge space 10.

During centrifuging The conical configuration of the liquid discharge space 10 guarantees that the liquid discharged through the liquid supply opening or respectively discharge opening cannot spray back onto the microscope slides 40.

1. A sample support washing container for simultaneous washing of at least two sample slides with microarrays or tissue slices, in particular microscope slides, the sample support washing container comprising the following:
a filling opening (2), through which the sample slides (40) can be introduced,
a liquid supply opening or discharge opening (4) substantially opposite the filling opening (2), and
sample slide holding devices (3), arranged in the washing container (1) such that they can keep introduced sample slides (40) away from one another and a liquid discharge space (10) remains between the introduced sample slides and the liquid supply opening or discharge opening (4).

2. The sample support washing container as claimed in claim 1, which is configured substantially cylindrical, with the filling opening (2) and liquid supply opening or discharge opening (4) on opposite cylinder end faces.

3. The sample support washing container as claimed in claim 2, wherein the sample slide holding devices (3) are arranged such that the sample slides are held substantially parallel to the cylinder axis.

4. The sample support washing container as claimed in claim 1, wherein the external dimensions correspond to the standard measurements for centrifuge tubes to be used in centrifuges, preferably 50 ml centrifuge tubes.

5. The sample support washing container as claimed in claim 1, wherein the sample support washing container interior tapers conically to the liquid supply opening or discharge opening (4).

6. The sample support washing container as claimed in claim 5, wherein the liquid discharge space (10) is formed by the inner volume of the conical termination (5).

7. The sample support washing container as claimed in claim 1, wherein the sample slide holders in the sample support washing container (1) comprise inwards pointing ribs (3).

8. The sample support washing container as claimed in claim 1, which comprises plastic as its material.

9. The sample support washing container as claimed in claim 1, wherein the inner measurements and the arrangement of the sample slide holders (3) are such that standard slide carriers (40) can be used.

10. A sample support washing station for washing sample slides with microarrays or tissue slices, in particular microscope slides, the sample support washing station comprising the following:
at least one take-up (6) for receiving a sample support washing container (1) as claimed in claim 1, the take-up (6) having a station supply/discharge (9), which corresponds to its liquid supply opening or discharge opening (4) when the sample support washing container (1) is removed,
an overflow vessel (7) arranged around the take-up (6),
a pump (13) connected to the station supply/discharge (9), and
at least one liquid supply valve (14, 16) attached to the pump (13).

11. The sample support washing station as claimed in claim 10, wherein the overflow vessel (7) comprises at least one discharge (8).

12. The sample support washing container as claimed in claim 10, wherein a valve block (14) is provided with several supply valves.

13. The sample support washing station as claimed in claim 10, with a liquid waste pipe branching off between the pump (13) and at least one supply valve (14).

14. The sample support washing station as claimed in claim 10, which comprises several take-ups (6).

15. The sample support washing station as claimed in claim 14, wherein the several take-ups (6) are enclosed by a common overflow vessel (7).

16. The sample support washing station as claimed in claim 10, wherein the pump comprises a hose pump (13).

17. The sample support washing station as claimed in claim 10, wherein the at least one take-up (6) is configured cylindrical to take-up a cylindrical sample support washing container (1).

18. The sample support washing station as claimed in claim 10, wherein the station supply/discharge (9) is configured conical.

19. The sample support washing station as claimed in claim 10, wherein the inner measurement of the at least one uptake area (6) corresponds to the outer measurement of a centrifuge tube which can be placed in centrifuges, preferably a 50 ml centrifuge tube.

20. The sample support washing container as claimed in claim 1, with a liquid supply opening or discharge opening (4), which corresponds to a station supply/discharge (9) of a takeup area (6) of a sample support washing station.

21. A system for washing sample slides with microarrays or tissue slices, in particular microscope slides, the system comprising a sample support washing station and at least one sample support washing container (1) as claimed in claim 1.

22. A method for washing and drying of sample slides with microarrays or tissue slices, in particular microscope slides, using at least one sample support washing container (1) and a sample support washing station claim, wherein
a) the sample slides (40) are placed in the sample support washing container (1),
b) the sample support washing container (1) is set in the take-up (6) of the sample support washing station,
c) at least one washing or rinsing liquid is rinsed through the station supply/discharge (9) of the take-up of the sample support washing station (6), the liquid supply opening or discharge opening (4) of the sample support washing container (1) and the sample support washing container (1),
d) the sample support washing container (1) after washing/rinsing with at least one washing or rinsing liquid is placed in a centrifuge, without the sample slides (40) being removed from the sample support washing container (1), and
e) the sample slides (1) are dried by centrifuging.

23. The method as claimed in claim 22, wherein first step b) and then steps a), c), d) and e) are carried out.

24. The method as claimed in claim 23, wherein on completion of step b) and prior to step a) a first washing/rinsing solution is introduced to the sample support washing container (1) via the station supply/discharge (9) of the take-up of the sample support washing station (6) and the liquid supply opening or discharge opening (4) of the sample support washing container (1).

25. The method for washing sample slides as claimed in claim 22, wherein different washing liquids with different washing times or temperatures are rinsed successively through the station supply/discharge (9) of the sample support washing station and the liquid supply opening or discharge opening (4) of the sample support washing container (1) and through the sample support washing container (1).

26. The method for washing sample slides as claimed in claim 22, wherein a mixing procedure is undertaken by briefly operating the pump (13).
